(19) United States
(12) Patent Application Publication
(10) Pub. No.: US 2002/0169395 A1
(43) Pub. Date: Nov. 14, 2002

(54) Urine collector for urinalysis

(76) Inventor: Tsu-Min Huang, Keelung (TW)

Correspondence Address: BACON & THOMAS, PLLC, 4th Floor, 625 Slaters Lane, Alexandria, VA, 22314-1176, US

(21) Appl. No.: 09/850,105

(22) Filed: May 8, 2001

(51) Int. Cl. A61B 5/00; B65D 81/00

(52) U.S. Cl. 600/574

(57) ABSTRACT

A urine collector for urinalysis includes a cup portion and a handle portion outward extended from an upper edge of the cup portion. A peripheral wall of the cup portion includes an inclined area downward and inward extended from a joint of the cup and the handle portions to a predetermined point, so that a distance exists between a lower end of the inclined area and a bottom of the cup portion. The peripheral wall below the lower end of the inclined area is bent to provide a stopper in front of the lower end of the inclined area. Pour spouts are provided on the upper edge of the cup portion to facilitate dispensing of urine collected in the cup portion. The handle portion enables convenient and sanitary holding of the urine collector. The stopper and the bottom of the cup portion provide two different depths of the cup portion relative to the upper edge thereof, adapting the urine collector to testing strips for routine urinalysis and pregnancy test.

### BACKGROUND OF THE INVENTION

The present invention relates to a urine collector for urinalysis, and more particularly to a urine collector that combines conventional cups for routine urinalysis and pregnancy test into one cup that could be handled in a convenient and sanitary manner.

It is known that urine is one of the most important sources of specimens for many kinds of medical tests to examine our organs and decide the health condition thereof. Currently, testing strips used in routine urinalysis and pregnancy test are differently structured. The testing strip for routine urinalysis has a reacting zone provided at a higher position of the strip while the testing strip for pregnancy test has a reacting zone provided at a lower position of the strip. As a result, a conventional urine-collecting cup 11 for routine urinalysis purpose, as shown in FIG. 1, is tall as compared with a conventional urine-collecting cup 12 for pregnancy test shown in FIG. 2. Moreover, the urine-collecting cup 12 for pregnancy test is further provided with at one side with an inclined area 13 extended from an upper edge to a bottom 15 of the cup 12 for disposing a test strip (not shown) thereat. A stopper 14 is formed on the bottom 15 at a lower end of the inclined area 13 to prevent the test strip at the inclined area from slipping off and resulting in an incorrect testing value.

The following are some of the disadvantages of the conventional urine-collecting cups 11 and 12:

1. The conventional urine-collecting cups 11 and 12 are intended for different purposes and could not be exchanged in conducting routine urine test and pregnancy test. It is not economical and inconvenient to produce two different types of urine-collecting cups while they all are used to collect urine specimens.

2. Moreover, both the conventional urine-collecting cups 11, 12 are not provided with pour spouts for dispensing the collected urine specimens. The medical personnel are subject to contamination by the urine during conducting tests. Spilt urine specimens also pollute the working environment to possibly result in undesired infection.

3. Users or medical personnel have to directly hold the cup 11 or 12 when collecting or dispensing urine specimens, and are therefore subject to the danger of contamination and infection due to contacting with the collected urine.

4. A urine-collecting cup without a handle is very inconvenient for use from the standpoint of a female user, particularly a pregnant female.

### SUMMARY OF THE INVENTION

It is therefore a primary object of the present invention to provide a urine collector for urinalysis that could be used for both a routine urinalysis and a pregnancy test.

Another object of the present invention is to provide a urine collector for urinalysis that is provided with a handle to facilitate convenient and sanitary collection of urine specimens without the risk of contacting with the collected urine.

A further object of the present invention is to provide a urine collector for urinalysis that is provided with pour spouts to facilitate convenient and sanitary dispensing of collected urine during conducting medical tests.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of a conventional urine collector in the shape of a cup for using in routine urinalysis;

FIG. 2 is a perspective view of another conventional urine collector in the shape of a cup for using in pregnancy test;

FIG. 3 is a perspective view of a urine collector for urinalysis according to a first embodiment of the present invention;

FIGS. 4a and 4b are sectioned side views of the urine collector of FIG. 3 illustrating the use thereof for pregnancy test and routine urinalysis, respectively;

FIG. 5 is an exploded perspective view of a urine collector according to another embodiment of the present invention; and

FIG. 6 is an assembled perspective view of the urine collector of FIG. 5.

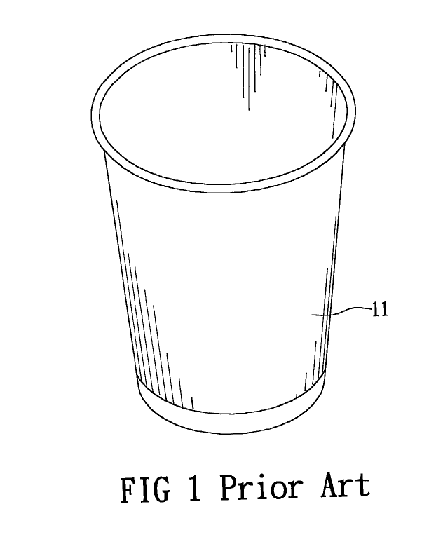

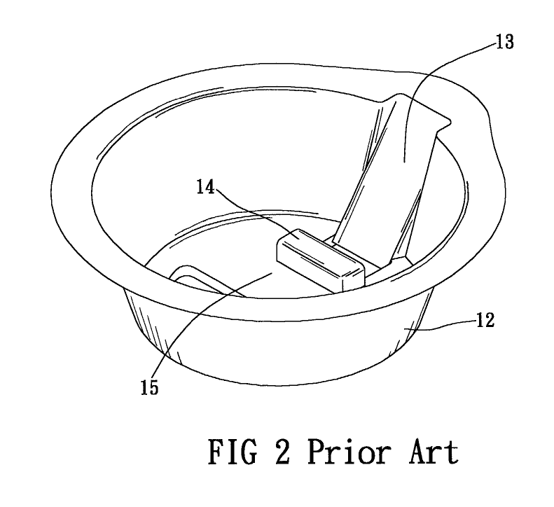

### DETAILED DESCRIPTION OF THE PREFERRED EMBODIMENTS

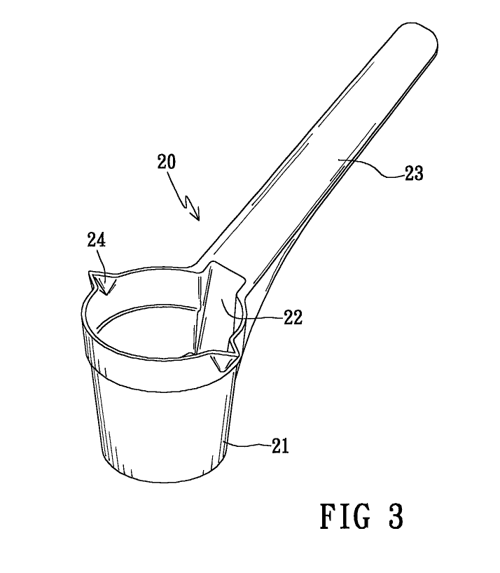

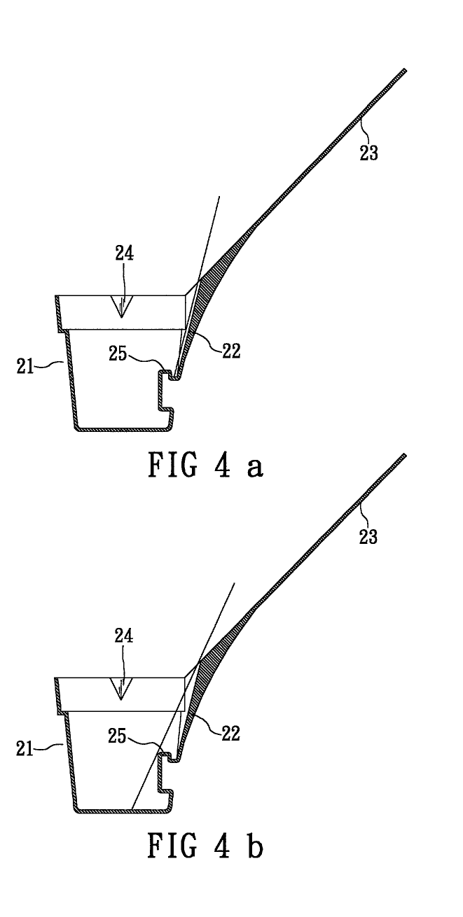

Please refer to FIGS. 3, 4a, and 4b that show a urine collector 20 for urinalysis according to a first embodiment of the present invention. As shown, the urine collector 20 mainly includes a cup portion 21 for containing an amount of urine specimen therein. The cup portion 21 has a depth suitable for testing the collected urine specimen directly with a testing strip. To enable convenient holding of the urine collector 20 without contacting with the collected urine, the cup portion 21 is provided at one side with an integrally formed handle portion 23 that upward and outward extends from an upper edge of the cup portion 21 at a predetermined angle. The cup portion 21 is also provided on the upper edge at predetermined positions with more than one pour spout 24 in, for example, a pyramidal shape to facilitate dispensing of collected urine without resulting in spilt urine.

For the urine collector 20 to adapt to testing strips used in different purposes, that is, for both routine urinalysis and pregnancy test, the cup portion 21 is formed at a wall thereof with a inclined area 22 that inward extends from a joint of the handle portion 23 and the upper edge of the cup portion 21 to a predetermined point of the wall, so that a distance exists between a lower end of the inclined area 22 and a bottom of the cup portion 21, as can be seen from FIGS. 4a and 4b. A portion of the wall of the cup portion 21 immediately below the lower end of the inclined area 22 is bent inward to form a recess and an adjacent projection that together provide a stopper 25 in front of the lower end of the inclined area 22. When the urine collector 20 of the present invention is used to collect urine for a pregnancy test, a medical testing personnel needs only to put a testing strip for this purpose into the inclined area 22 of the cup portion 21 with a lower end of the testing strip locating against the stopper 25, as shown in FIG. 4a. And, when the urine collector 20 is used to collect urine for a routine urinalysis, the medical testing personnel needs only to put a testing strip for this purpose into the inclined area 22 with the lower end of the testing strip locating on the bottom of the cup portion 21, as shown in FIG. 4b. In brief, with the stopper 25, the urine collector 20 of the present invention provides two different depths relative to the upper edge of the cup portion 21 to hold testing strips for two different types of tests.

The urine collector 20 with the above-described structure not only integrates the conventional urine collectors for routine urinalysis and pregnancy test, but also enables users and/or medical personnel to handle the urine specimen in the urine collector in a convenient and sanitary manner.

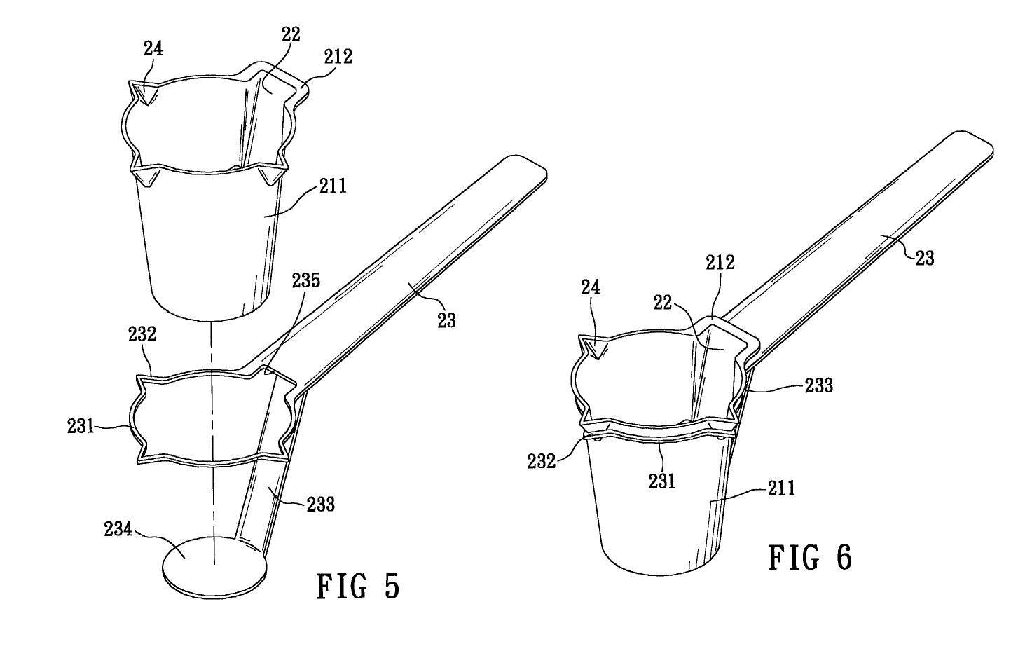

FIGS. 5 and 6 are exploded and assembled perspective views, respectively, of a urine collector according to a second embodiment of the present invention particularly designed to reduce unnecessary discard and meet the economical requirement and environment protection. In this second embodiment, the urine collector includes separate cup portion 211 and handle portion 23 that could be assembled to each other as necessary. The handle portion 23 is provided at a front end, that is, an end closer to the cup portion 211, with a frame 231 into which the cup portion 211 is removably positioned. A notch 235 is provided between the frame 231 and the handle portion 23 corresponding to an inclined area 22 provided on a wall of the cup portion 211, so that a top portion of the inclined area 22 is fitted in the notch 235 when the cup portion 211 is disposed in the frame 231. The top portion of the inclined area 22 is provided with an outward extended flange 212 for resting on edges of the notch 235, so that the inclined area 22 is stably supported on the front end of the handle portion 23. The handle portion 23 also includes an arm portion 233 that downward and forward extends from a lower front of the handle portion 23 closely behind the notch 235 at an angle corresponding to an inclination of the inclined area 22. A lower end of the arm portion 233 is a forward extended horizontal bottom plate 234 for supporting the cup portion 211 thereon.

The frame 231 may be provided with outward projections 232 corresponding to pyramidal pour spouts 24 provided on an upper edge of the cup portion 211, so that the cup portion 211 is more securely held in the frame 231.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A urine collector for urinalysis, comprising integrally formed cup portion and handle portion;
said cup portion being provided on an upper edge with more than one pour spout to facilitate dispensing of urine collected in said cup portion, and at one side of a peripheral wall with an inclined area downward and inward extended from the upper edge to a predetermined point of the peripheral wall, so that a distance exists between a lower end of said inclined area and a bottom of said cup portion; a portion of said peripheral immediately below said lower end of said inclined area being bent inward to form a recess and an adjacent projection that together provide a stopper in front of the lower end of said inclined area; and said stopper and said bottom of said cup portion providing said urine collector with two different depths relative to the upper edge of said cup portion, adapting said urine collector to testing strips for routine urinalysis and pregnancy test; and
said handle portion being outward extended from the upper edge of said cup portion to facilitate convenient and sanitary holding of said urine collector.

2. The urine collector for urinalysis as claimed in claim 1, wherein said pour spout is pyramidal in shape.

3. A urine collector for urinalysis, comprising separable cup portion and handle portion; said cup portion being provided on an upper edge with more than one pour spout to facilitate dispensing of urine collected in said cup portion, and at one side of a peripheral wall thereof with an inclined area downward and inward extended from the upper edge to a predetermined point of the peripheral wall, so that a distance exists between a lower end of said inclined area and a bottom of said cup portion; a portion of said peripheral wall immediately below the lower end of said inclined area being bent inward to form a recess and an adjacent projection that together provide a stopper in front of the lower end of said inclined area; and said stopper and said bottom of said cup portion providing said urine collector with two different depths relative to the upper edge of said cup portion, adapting said urine collector to testing strips for routine urinalysis and pregnancy test; and
said handle portion being provided at a front end, that is, an end closer to said cup portion, with a frame that has an inner diameter smaller than an outer diameter of the upper edge of said cup portion for removably holding said cup portion therein, a portion between said frame and said handle portion being formed of a notch corresponding to said inclined area provided on said cup portion, so that said cup portion is positioned in said frame with a top portion of said inclined area located in said notch.

4. The urine collector for urinalysis as claimed in claim 3, wherein said inclined area is provided along the top portion with an outward extended flange for resting on outer edges of said notch on said handle portion.

5. The urine collector for urinalysis as claimed in claim 3, wherein said handle portion includes an arm portion downward extended from a lower front of said handle portion closely behind said notch, and a lower end of said arm portion being provided with a forward projected horizontal bottom plate for supporting said cup portion thereon.

6. The urine collector for urinalysis as claimed in claim 3, wherein said frame of said handle portion is provided with outward projections corresponding to said pour spouts on the upper edge of said cup portion.
